(19) United States
(12) Patent Application Publication
(10) Pub. No.: US 2024/0207503 A1
(43) Pub. Date: Jun. 27, 2024

(54) Anti-Twist Phacoemulsification Tip

(71) Applicant: JOHNSON & JOHNSON SURGICAL VISION, INC., Irvine, CA (US)

(72) Inventors: Vadim Gliner, Haifa (IL); Alon Boumendil, Givat Nili (IL)

(21) Appl. No.: 18/089,389

(22) Filed: Dec. 27, 2022

(51) Int. Cl. A61M 1/00

(52) U.S. Cl. CPC A61M 1/85; A61B 2017/320084

(57) ABSTRACT

A phacoemulsification probe includes an irrigation sleeve configured to fit over a hollow aspiration needle and a probe body, the irrigation sleeve to provide a fluid pathway for the flow of irrigation fluid between the needle and the walls of the irrigation sleeve, the sleeve having a distal end insertable into an incision made in an eye, the distal end having ports to release said irrigation fluid into said eye, where the walls of the irrigation sleeve have at least one anti-tortional element longitudinally placed along a length of the irrigation sleeve to provide structural support to prevent twisting of the sleeve and where the at least one anti-tortional element allows flattening of the irrigation sleeve for maneuvering of the sleeve within the eye while preserving the integrity of the incision.

### FIELD OF THE INVENTION

The present invention relates to cataract surgery generally and to phacoemulsification in particular.

### BACKGROUND OF THE INVENTION

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery.

In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution (BSS) to maintain the anterior chamber of the eye. After removing the cataract with phacoemulsification, the softer outer lens cortex is removed with suction. An intraocular lens (IOL) is then introduced into the empty lens capsule restoring the patient's vision.

Reference is made to FIG. 1 and FIG. 2 which illustrate a phacoemulsification probe 10 as described in U.S. patent application Ser. No. 17/511,166 filed 26 Oct. 2021) commonly owned by the Applicant Phacoemulsification probe 10 comprises a probe body 17 (the area that is typically held by the person performing surgery), and a coaxial irrigation sleeve 56 that at least partially surrounds needle 16 and creates a fluid pathway between the wall of needle 16 and the wall 561 of irrigation sleeve 56. Needle 16 is hollow to provide an aspiration channel.

During the phacoemulsification procedure, a pumping subsystem (not shown) pumps irrigation fluid from an irrigation reservoir to the irrigation sleeve 56 to irrigate eye 20. The irrigation fluid is pumped to an irrigation channel 45 of body 17 through to sleeve 56 and out through ports 71 at the distal end 12 of sleeve 56 into eye 20 as is illustrated in FIG. 2.

Eye fluid and waste matter (e.g. emulsified parts of the cataract) are aspirated via an aspiration channel 47, which extends from the hollow of needle 16 through the body 17, and then via an aspiration tubing line 46 to a collection receptacle. The aspiration is affected by a pumping sub-system (not shown).

It is typically desired to control the irrigation and aspiration flow in a manner that maintains a steady IOP (intraocular pressure).

### SUMMARY OF THE PRESENT INVENTION

There is provided, in accordance with a preferred embodiment of the present invention, phacoemulsification probe. The phacoemulsification probe includes an irrigation sleeve configured to fit over a hollow aspiration needle and a probe body, the irrigation sleeve to provide a fluid pathway for the flow of irrigation fluid between the needle and walls of the irrigation sleeve, the sleeve having a distal end insertable into an incision made in an eye, the distal end having ports to release the irrigation fluid into the eye, where the walls of the irrigation sleeve have at least one anti-tortional element longitudinally placed along a length of the sleeve to provide structural support to prevent twisting of the sleeve and where the at least one anti-tortional element allows flattening of the irrigation sleeve for maneuvering of the sleeve within the eye while preserving the integrity of the incision.

Moreover, in accordance with a preferred embodiment of the present invention, the at least one anti-tortional element is embedded in the walls.

Further, in accordance with a preferred embodiment of the present invention, the at least one anti-tortional element is affixed to the outside of the walls.

Still further, in accordance with a preferred embodiment of the present invention, the at least one anti-tortional element forms a spiral around the irrigation sleeve.

Additionally, in accordance with a preferred embodiment of the present invention, the irrigation sleeve is manufactured from silicon.

Moreover, in accordance with a preferred embodiment of the present invention, the at least one anti-tortional element is manufactured from metal.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a phacoemulsification probe:

FIG. 2 is a schematic illustration of a closeup of the phacoemulsification probe of FIG. 1 in use:

FIG. 3 is a schematic illustration of the phacoemulsification probe of FIG. 1 in a twisted position during use:

FIG. 4 is a schematic illustration of the phacoemulsification probe of FIG. 1 with anti-tortional elements: constructed and operative in accordance with a preferred embodiment of the present invention:

FIG. 5 is a schematic illustration of an alternative embodiment to the phacoemulsification probe of FIG. 4, constructed and operative in accordance with a preferred embodiment of the present invention; and

FIG. 6 is a schematic illustration of an alternative embodiment to the phacoemulsification probe of FIG. 4: constructed and operative in accordance with a preferred embodiment of the present invention.

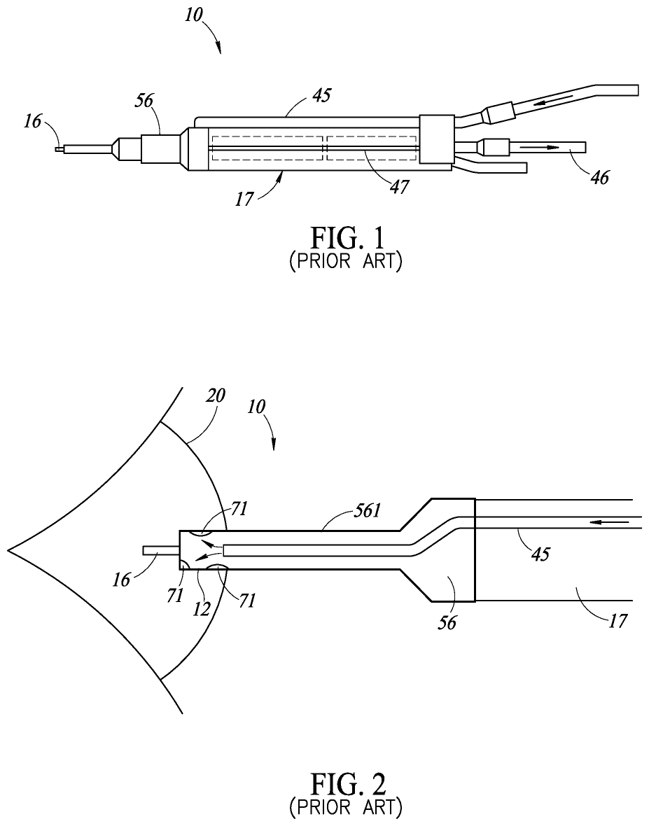

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

One advantage of phacoemulsification is that it provides performing the cataract procedure with only a small incision made to the cornea. To enable the tip together with a distal end of the irrigation sleeve to be easily inserted into the incision without applying a significant amount of strain on the tissue surrounding the incision, it is advantageous to form the irrigation sleeve from a flexible material. In this manner, the sleeve can flatten to take on the shape of the incision when inserted into the surgical site and avoid excessive strain on tissue surrounding the incision potentially making it bigger.

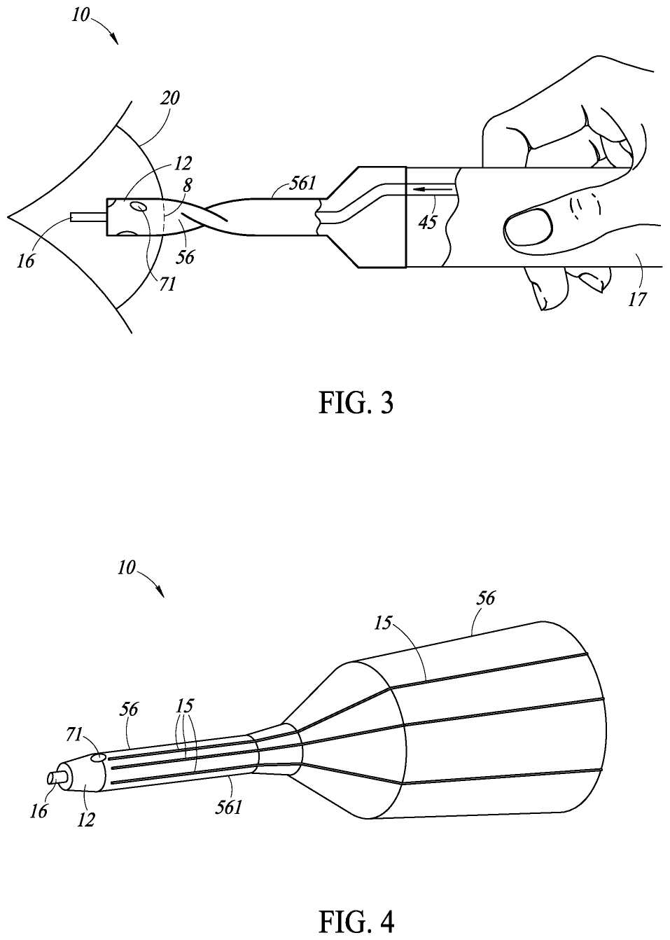

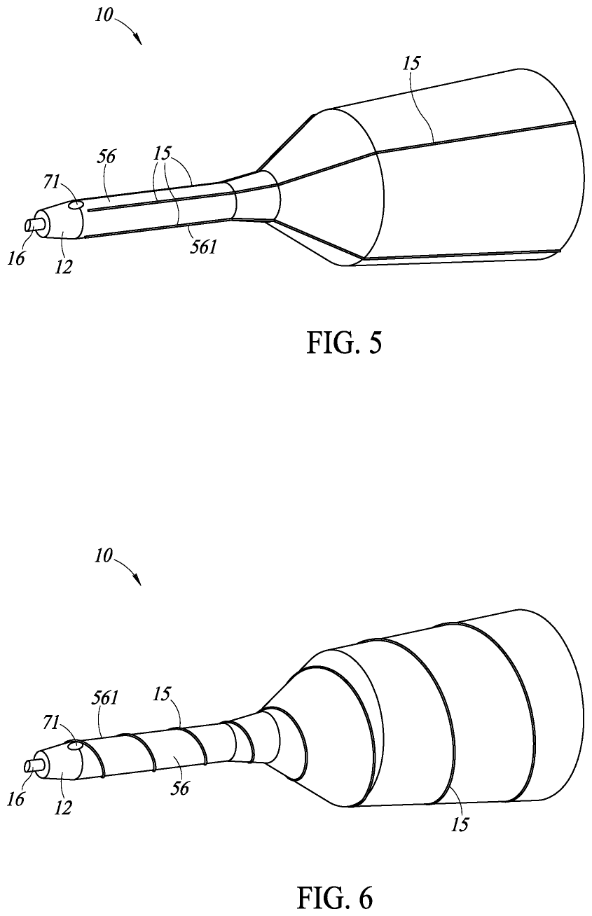

Applicant has also realized that during a phacoemulsification procedure, a physician may turn probe 10 about its longitudinal axis to maneuver it. This rotational motion may at times lead to twisting of sleeve 56 as shown in FIG. 3 to which reference is now made. The twisting may occur due to the tacky composition of the tissue surrounding incision 8. The tissue tends to grip sleeve 56 at the location of the incision. Twisting of sleeve 56 may block the outflow of irrigation through ports 71 and impede the exit of the irrigation fluid which may then build up in sleeve 56. The obstruction may lead to imbalance in the IOP that can cause suction of the eye during operation of the aspiration flow.

Applicant has realized that while it is possible to reduce the overall flexibility of irrigation sleeve 56 to prevent twisting, reducing the overall flexibility may also reduce the ability of sleeve 56 to respond to pressure applied by the tissue surrounding incision 8 by flattening. Applicant has realized that it is preferable to prevent the tendency of sleeve 56 to twist without preventing its ability to flatten. As stated herein above, the sleeve 56 to take on the shape of incision 8 when inserted into the surgical site, avoiding excessive strain on tissue surrounding incision 8. It will also be appreciated that although a flattened sleeve 56 may decrease the cross-sectional area of sleeve 56, it will typically not lead to a block in the irrigation flow since the presence of needle 16 may prevent total collapse.

Prior art solutions use rigid materials for the walls of the sleeve and/or the addition of internal ribs to prevent circumferential vibrations. These materials and ribs may also add rigidity to the sleeve accompanied by resistance to flattening when inserted into the incision. Applicant has further realized that these solutions may prevent collapsing (and potential twisting) but may also prevent any flexibility for insertion of the probe into the eye and preservation of the integrity of incision 8.

According to an exemplary embodiment of the present invention, there is provided an irrigation sleeve 56 that is structured to resist twisting without significantly reducing the tendency of irrigation sleeve 56 to flatten based on pressure applied by the tissue surrounding incision 8. In this manner blockage of the irrigation flow may be prevented without compromising the integrity of incision 8, i.e. without significantly increasing the size of the original incision made.

Sleeve 56 may be manufactured from silicon or any elastomeric material. In some example embodiments, the walls 561 of sleeve 56 may be thin to be flexible enough to allow for flattening against pressure applied by the tissue surrounding the incision.

Anti-tortional elements 15 may be added to walls 561 of sleeve 56 as is illustrated in FIG. 4 to which reference is now made. It will be appreciated that the combination of anti-tortional elements 15 and the material of walls 561 may still allow flattening during insertion into eye 20, but may prevent twisting of sleeve from occurring as described herein above.

In some example embodiments, anti-tortional elements 15 may be formed from metal. Optionally, anti-tortional elements 15 may be metal wires or flat strips that are embedded in walls 561. In other examples they may be flat strips or (ribbons) embedded in or otherwise affixed onto walls 561. In yet other examples, external anti-tortional elements 15 and are integral to sleeve 56. It will be appreciated that for all the above mentioned embodiments, anti-torsional elements 15 may extend longitudinally along the length of sleeve 56. The spring effect of the metal may allow for the flexible structural support against twisting. In other embodiments, anti-tortional elements 15 may by formed from silicon or any other flexible material.

In yet a further embodiment, as is illustrated in FIG. 6 to which reference in now made anti-torsional element 15 may be a metal coil that is embedded or otherwise fixed to sleeve 56, with the axis of the coil extending along the length of sleeve 56.

It will be appreciated that for all the above mentioned embodiments, anti-tortional elements 15 do not affect the available cross-sectional area of sleeve 56 for the required irrigation flow.

Thus the phacoemulsification probe 10 may undergo rotation and/or vibration during the phacoemulsification process and the anti-tortional elements 15 of sleeve 56 may resist torsion and may also reduce the friction between the tissue around the incision and the sleeve allowing for collapsing but preventing sleeve 56 from twisting and therefore preserving the integrity of the incision made in eye 20.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A phacoemulsification probe comprising:
an irrigation sleeve configured to fit over a hollow aspiration needle and a probe body, said irrigation sleeve to provide a fluid pathway for the flow of irrigation fluid between said needle and walls of said irrigation sleeve, said sleeve having a distal end insertable into an incision made in an eye, said distal end having ports to release said irrigation fluid into said eye;
wherein the walls of said irrigation sleeve have at least one anti-tortional element longitudinally placed along a length of said sleeve to provide structural support to prevent twisting of said sleeve; and
wherein said at least one anti-tortional element allows flattening of said irrigation sleeve for maneuvering of said sleeve within said eye while preserving the integrity of said incision.

2. The phacoemulsification probe of claim 1 wherein said at least one anti-tortional element is embedded in said walls.

3. The phacoemulsification probe of claim 1 wherein said at least one anti-tortional element is affixed to the outside of said walls.

4. The phacoemulsification probe of claim 2 wherein said at least one anti-tortional element forms a spiral around said irrigation sleeve.

5. The phacoemulsification probe of claim 3 wherein said at least one anti-tortional element forms a spiral around said irrigation sleeve.

6. The phacoemulsification probe of claim 1 wherein said irrigation sleeve is manufactured from silicon.

7. The phacoemulsification probe of claim 1 wherein said at least one anti-tortional element is manufactured from metal.
